(19) United States
(12) Patent Application Publication
(10) Pub. No.: US 2026/0283691 A1
(43) Pub. Date: Sep. 24, 2026

(54) LASER DEBULKING DEVICES

(71) Applicant: Bard Peripheral Vascular, Inc., Franklin Lakes, NJ (US)

(72) Inventors: German Loesener, Saratoga Springs, UT (US); Zachary Willis, Mesa, AZ (US); Carter Valentino Righi, Mesa, AZ (US); Joseph Michael Addante, Phoenix, AZ (US)

(21) Appl. No.: 19/086,418

(22) Filed: Mar. 21, 2025

(51) Int. Cl. A61B 18/24; A61B 17/00; A61B 18/00; A61B 18/22

(52) U.S. Cl. CPC A61B 18/245; A61B 2017/00287; A61B 2018/00214; A61B 2018/0041; A61B 2018/2288; A61B 2218/007

(57) ABSTRACT

An example laser debulking device includes a catheter having a proximal end region and a distal end region opposite the proximal end region. The laser debulking device also includes a plurality of fingers integrally formed with (i.e., exhibit single piece construction with) or are attached to the distal end region. The fingers are configured to switch between a deployment state and an expanded state, wherein a maximum lateral dimension of the fingers is greater when the fingers are in the expanded state than the deployment state. The laser debulking device also includes a plurality of laser emitters (e.g., the terminal ends of optical fibers). At least some of the fingers includes at least one of the laser emitters.

### BACKGROUND

Conventional debulking systems are configured to remove material from a vascular structure. Conventional debulking systems may include physical debulking systems (e.g., debulking systems including a rotating drill-like structure) or lasers. However, these conventional debulking systems may be unable to remove substantially all of the desired material from the vascular structure, may damage the vascular structure, or may be only able to remove the desired material in very specific applications.

### SUMMARY

Embodiments disclosed herein related to laser debulking devices, laser debulking systems including the same, and methods of using the same. In an embodiment, a laser debulking device is disclosed. The laser debulking device includes a catheter including a proximal end region and a distal end region opposite the proximal end region. The laser debulking device also includes a plurality of fingers integrally formed with or attached to the distal end region. The plurality of fingers are configured to switch between a deployment state and an expanded state. A maximum lateral dimension of the plurality of fingers is greater when in the expanded state than the deployment state. The laser debulking system also includes a plurality of laser emitters. At least some of the plurality of fingers include at least one of the plurality of laser emitters.

In an embodiment, a laser debulking system is disclosed. The laser debulking system includes a laser debulking device. The laser debulking device includes a catheter including a proximal end region and a distal end region opposite the proximal end region. The laser debulking device also includes a plurality of fingers integrally formed with or attached to the distal end region. The plurality of fingers are configured to switch between a deployment state and an expanded state. A maximum lateral dimension of the plurality of fingers is greater when in the expanded state than the deployment state. The laser debulking system also includes a plurality of laser emitters. At least some of the plurality of fingers include at least one of the plurality of laser emitters. The laser debulking system includes a sheath defining interior passageway. The catheter of the laser debulking device is at least partially disposed in the interior passageway. The sheath is configured to slide along the catheter. The plurality of fingers are in the deployment state when the plurality of fingers are disposed in the interior passageway and are in the expanded state when the plurality of fingers are at least partially disposed outside of the interior passageway.

In an embodiment, a method of using a laser debulking device is disclosed. The method includes positioning a distal end region of a catheter adjacent to or through a selected material to be debulked. The selected material to be debulked is located within a vascular structure. The method also includes switching a plurality of fingers from a deployment state to an expanded state. The plurality of fingers attached to or forming the distal end region. The method further includes, with the plurality of fingers in the expanded state, emitting a plurality of laser pulses from a plurality of laser emitters towards the selected material to be debulked, at least some of the plurality of fingers including at least one of the plurality of laser emitters.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIGS. 1A and 1B are cross-sectional views of a portion of a laser debulking system in two different configurations, according to an embodiment.

FIG. 1C is a front plan view of the laser debulking system in the expanded configuration.

FIGS. 2A and 2B are cross-sectional views illustrating a method of using the laser debulking system, according to an embodiment.

FIGS. 3A, 3B, and 3C are cross-sectional views of a laser debulking system in the deployment and expanded states, respectively, according to an embodiment.

FIGS. 4A and 4B are cross-sectional views illustrating a method of using the laser debulking system, according to an embodiment.

FIG. 5 is a front view of a laser debulking device including one or more aspiration channels, according to an embodiment.

FIG. 6 is a side view of a laser debulking device including a capture bag, according to an embodiment.

FIG. 7 is a schematic side view of a laser debulking system, according to an embodiment.

### DETAILED DESCRIPTION

Embodiments disclosed herein related to laser debulking devices, laser debulking systems including the same, and methods of using the same. An example laser debulking device includes a catheter having a proximal end region and a distal end region opposite the proximal end region. The laser debulking device also includes a plurality of fingers integrally formed with (i.e., exhibit single piece construction with) or are attached to the distal end region. The fingers are configured to switch between a deployment state and an expanded state, wherein a maximum lateral dimension of the fingers is greater when the fingers are in the expanded state than the deployment state. The laser debulking device also includes a plurality of laser emitters (e.g., the terminal ends of optical fibers). At least some of the fingers includes at least one of the laser emitters.

During use, the laser debulking device is inserted into a vascular structure (e.g., artery, vein, valve, etc.) of an individual. The laser debulking device may be positioned in the vascular structure such that the distal end region of the catheter is positioned adjacent to a selected material or extends through the selected material. As used herein, the “selected material” refers to a material to be debulked and may include, for example, a thrombus, scar tissue, an occlusion of an occluded venous stent, a venous stent (e.g., the laser debulking device deanchors and removes or repositions the venous stent, such as a smaller length venous stent), material received by or built up around an inferior vena cava filter, or any other material which may be desirable to be debulked and removed from the vascular structure. With the laser debulking device positioned, the fingers of the debulking device may be switched from a deployment state (which facilitates insertion and positioning of the laser debulking device in the vascular structure and can be at least partially collapsed) to the expanded state. For example, the fingers may expand until the fingers exhibit a maximum lateral dimension that is generally equal to the maximum lateral dimension of the vascular structure. After expanding the fingers, a plurality of laser pulses may be emitted from the laser emitters. Since the fingers exhibit a maximum lateral dimension that is generally equal to the maximum lateral dimension of the vascular structure, the laser pulses may irradiate and damage the selected material at or near the boundary between the walls of the vascular structure and the selected material thereby allowing the laser debulking device to remove all of the selected material from the vascular structure.

The debulking device is an improvement over conventional laser debulking devices. An example of a laser debulking device is the CavaClear, available from Philips®. This conventional laser debulking device includes laser emitters in an annular arrangement. However, the maximum lateral dimension of the laser emitters of this conventional laser debulking device does not change. As such, this conventional laser debulking device struggles to remove the selected material from the boundary between the walls of the vascular structure unless the vascular structure exhibits a maximum lateral dimension equal to the maximum lateral dimension of the annular arrangement of the laser emitters. Further, this conventional laser debulking device may only be used to remove tissue buildup around an inferior vena cava filter. The laser debulking device disclosed herein is an improvement over this conventional laser debulking device by allowing the maximum lateral dimension of the fingers to change which permits the laser debulking device to be used with vascular structures exhibiting various sizes. Further, the laser debulking devices disclosed herein may be used with selected material other than tissue buildup around an inferior vena cava filter.

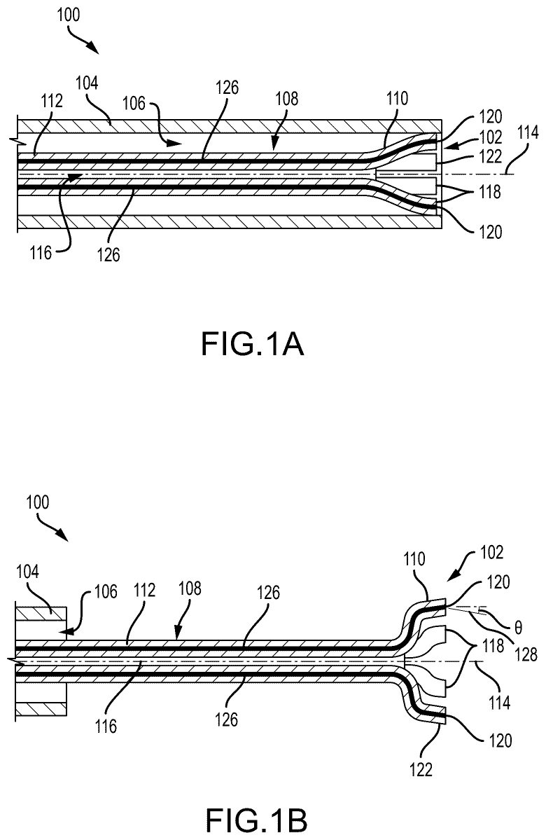

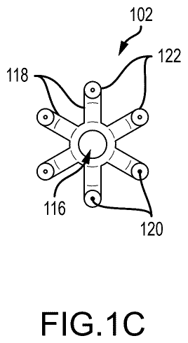

FIGS. 1A and 1B are cross-sectional views of a portion of a laser debulking system 100 in two different configurations (i.e., a deployment and expanded configuration, respectively), according to an embodiment. FIG. 1C is a front plan view of the laser debulking system 100 in the expanded configuration. The laser debulking system 100 includes a laser debulking device 102 and a sheath 104. The sheath 104 defines an interior passageway 106 and the laser debulking device 102 is at least partially disposed in the interior passageway 106.

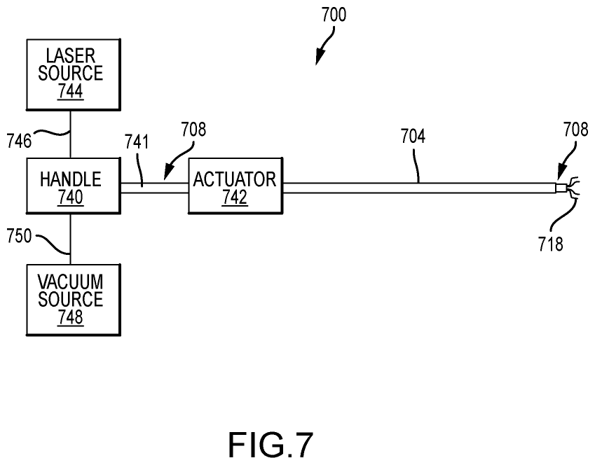

The laser debulking device 102 includes a catheter 108. The catheter 108 includes a proximal end region (not shown), a distal end region 110 opposite the proximal end region, and a central region 112 (partially shown in FIGS. 1A and 1B) extending between the proximal end region and the distal end region 110. The catheter 108 may be configured to extend longitudinally away from a handle (e.g., handle 740 of FIG. 7) of a laser debulking system 100. For example, the proximal end region is configured to be attached to, disposed in, or otherwise near the handle. The catheter 108 may then extend longitudinally away from the handle in parallel to a central longitudinal axis 114 of the catheter 108. It is noted that the catheter 108 may not be linear and, as such, the central longitudinal axis 114 may not be linear and, instead, may follow the path of the catheter 108.

The catheter 108 may define a central passageway 116. In an embodiment, the central passageway 116 may be configured to receive a guidewire. In such an embodiment, during use of the laser debulking system 100, the guidewire may be inserted into the individual and advanced into the individual until the guidewire reaches or passes the selected material. The catheter 108 may then be positioned around the guidewire (e.g., the guidewire is positioned in the central passageway 116) and then advanced along the guidewire until the catheter 108 is adjacent or passes through the selected material. In an embodiment, the central passageway 116 may receive medical devices other than or in addition to the guidewire, such as an intravenous ultrasound device. In an embodiment, the central passageway 116 may form an aspiration channel that provides suction to the area about the distal end region 110.

The laser debulking device 102 includes a plurality of fingers 118. In an embodiment, as shown, the plurality of fingers 118 form the distal end region 110 of the catheter 108. In such an embodiment, the fingers 118 are integrally formed (e.g., exhibit single piece construction) with at least a portion of the central region 112 adjacent to the distal end region 110. In an embodiment, the fingers 118 may be attached to the distal end region 110. In such an embodiment, the fingers 118 may be attached to the distal end region 110 using any suitable technique. For example, the fingers 118 may form an interference fit with, be adhesive attached to, be welded to, or be otherwise attached to the distal end region 110.

The fingers 118 are configured to switch between a deployment state and an expanded state. The fingers 118 exhibit a maximum lateral dimension that is greater when the fingers 118 are in the expanded state than when the fingers 118 are in the deployment state. As used herein, the maximum lateral dimension of the fingers 118 refers to the maximum lateral dimension measured between opposing fingers 118 or a diameter of a circle forming an outer periphery of the fingers 118, whichever is greater. The smaller maximum lateral dimension of the fingers 118 may facilitate insertion of the laser debulking device 102 into an individual and moving the laser debulking device 102 in the vascular structure. The larger maximum lateral dimension of the fingers 118, which may be about 10 mm to about 30 mm (e.g., 12 mm to about 25 mm), may facilitate removal of the selected tissue from the vascular structure. For example, switching the fingers 118 from the deployment state to the expanded state may cause the fingers 118 to expand until the fingers 118 abut the vascular structure. In other words, the maximum lateral dimension of the fingers 118 may expand until the maximum lateral dimension corresponds to the maximum lateral dimension of the vascular structure. As will be discussed in more detail below, at least some of the fingers 118 may include laser emitters 120 that are configured to emit laser pulses 128 (one laser pulse 128 shown in FIG. 1B). The fingers 118 exhibiting a maximum lateral dimension that corresponds to the maximum lateral dimension of the vascular structure allows the laser pulses 128 to be directed at a region of the selected tissue at or near the walls of the vascular structure thereby allowing the laser pulses 128 to remove substantially all of the selected tissue from the vascular structure.

The maximum lateral dimension of the fingers 118 may be changed using any suitable technique. FIGS. 1A and 1B illustrate an embodiment of a technique that may be used to switch the fingers 118 between the collapses and expanded states. For example, FIG. 1A illustrates the fingers 118 in the deployment state. When the fingers 118 are in the deployment state, the fingers 118 may exhibit a maximum lateral dimension that is similar (e.g., ±50% and, more particularly ±25% or ±10%) of the maximum lateral dimension of the central region 112 of the catheter 108. Such a maximum lateral dimension of the fingers 118 allows the fingers 118 to be inserted into the individual and moved within the vascular structure substantially as easily as a substantially similar catheter that did not include the fingers 118. To maintain the fingers 118 in the deployment state, the laser debulking system 100 may include a sheath 104, as previously discussed. The sheath 104 may exhibit an 8 French to 20 French size, such as a 10 French to 16 French size. The fingers 118 may be at least partially disposed in the interior passageway 106. As such, the sheath 104 limits expansion of the fingers 118 and maintains the fingers 118 in the deployment state. The sheath 104 may be configured to move (e.g., slide) along the catheter 108. For example, the sheath 104 may slide in a proximal direction (i.e., the direction extending along a central longitudinal axis 114 of the catheter 108 from the distal end region 110 to the proximal end region) or a distal direction (i.e., the direction extending along a central longitudinal axis 114 of the catheter 108 from the proximal end region to the distal end region 110). Moving the sheath 104 in the proximal direction may cause the fingers 118 to be at least partially outside of the interior passageway 106 such that the fingers 118 are at least partially not covered by the sheath 104 which, in turn, allows the fingers 118 to expand. In other words, moving the sheath 104 in the proximal direction may switch the fingers 118 from the deployment state to the expanded state. FIGS. 1B and 1C illustrate the fingers 118 in the expanded state. It is noted that the fingers 118 may be switched from the expanded state to the deployment state. For example, moving the sheath 104 in the distal direction may force the fingers 118 to enter the interior passageway 106. In such an example, the sheath 104 applies a compressive force to the fingers 118 that causes the maximum lateral dimension of the fingers 118 to decrease. It is noted that the fingers 118 may switch between the deployment and expanded states using technique other than or in addition to the sheath 104 moving distally and proximally. In an example, the fingers 118 may include a shape memory material that may change from deformed shape (e.g., a collapsed or expanded state) to a remembered shape (e.g., the other of the collapsed or expanded state). In an embodiment, the fingers 118 are maintained in or switched between the deployment and expanded states using other means. For example, the fingers 118 may be controlled, either individually or as a group, from the proximal end of the catheter 108 (e.g., from the handle 740 shown in FIG. 7) using a mechanical or electrical controller.

When in the expanded state, the fingers 118 may face the distal direction. That is, a terminal end 122 of the fingers 118 may generally face in the distal direction. When the terminal end 122 of the fingers 118 include the laser emitters 120, the fingers 118 facing the distal direction allows the laser emitters 120 to emit the laser pulses 128 in the distal direction. As such, during use, the laser debulking device 102 may be positioned in the vascular structure such that the selected material is positioned distally to the fingers 118.

The fingers 118 may be formed from any suitable biocompatible material. In an example, the fingers 118 may be formed from a nickel-titanium alloy (e.g., nitinol). The nickel-titanium alloy may exhibit one or more properties that allows the fingers 118 to switch from the deployment state to the expanded state. In an instance, the nickel-titanium alloy may exhibit superelasticity. In such an instance, the relaxed shape of the fingers 118 (e.g., the shape of the fingers 118 when no external force is applied thereof) may correspond to the expanded-most state of the fingers 118. The superelasticity of the nickel-titanium alloy may allow the fingers 118 to be plastically compressed into the deployment state (e.g., using the sheath 104) such that removal of the compressive force allows the fingers 118 to spring into the expanded state. In an example, the nickel-titanium alloy may exhibit a shape-memory effect. In such an instance, the “remembered” shape of the fingers 118 may correspond to the expanded-most state of the fingers 118. The nickel-titanium alloy of the fingers 118 may be configured such that the bodily heat of the individual may cause the fingers 118 to switch from the deformed shape to the remembered shape. Thus, disposing the fingers 118 in the body and permitting the fingers 118 to expand (e.g., the sheath 104 does not cover the fingers 118) causes the fingers 118 to switch from the deployment state to the expanded state. In an example, the fingers 118 may be formed of a material other than or in addition to a nickel-titanium alloy, such as stainless steel, titanium, other biocompatible metals, silicone, other biocompatible polymers, or combinations thereof. In such an example, the laser debulking device 102 may include a spring that applies a force to the fingers 118 that causes the fingers 118 to preferentially be in the expanded state when the fingers 118 are not covered by the sheath 104.

The laser debulking device 102 may include any suitable number of fingers 118. For example, the laser debulking device 102 may include 2, 3, 4, 5, 6, 7, 8, 9, 10, 11, 12, 13, 14, 15, or 16 or more fingers 118. Generally, increasing the number of fingers 118 allows the laser debulking device 102 to remove the selected material more efficiently and may minimize any rotation of the laser debulking device 102 that is needed to remove the selected material. However, the number of fingers 118 that may be included in the laser debulking device 102 may be limited for one or more reasons. For instance, the size of the interior passageway 106 of the sheath 104 may limit the number of fingers 118 that may be disposed therein. In another instance, the laser source (e.g., laser source 744 of FIG. 7) may only be able to provide laser pulses 128 to a certain number of laser emitters 120 which, in turn, may limit the number of fingers 118.

Generally, due to size constraints, the laser source that generates the laser pulses 128 is not disposed in the fingers 118 and, instead, is spaced from the fingers 118. As such, the laser debulking device 102 may include one or more devices that are configured to transport the laser pulses 128 from the laser source to the fingers 118 and, more particularly, to the laser emitters 120. In an embodiment, as shown, the laser debulking device 102 includes one or more fiber optic cables 126 that are configured to transport the laser pulses 128 to the laser emitters 120. The fiber optic cables 126, for instance, may extend at least from the proximal end region to the distal end region 110 when the laser source is external to the laser debulking device 102. Although the present application primarily discusses using the fiber optic cables 126 to transport the laser pulses 128, it is noted that other devices may be used instead of or in addition to the fiber optic cables 126, such as lenses or a void spaced (e.g., a void spaced defined by a reflective material).

In an embodiment, as shown, at least a portion of the fiber optic cables 126 may be disposed within the walls of the catheter 108 and the fingers 118. Disposing the fiber optic cables 126 within the walls of the catheter 108 and the fingers 118 may allow the walls of the catheter 108 and the fingers 118 to protect the fiber optic cables 126 from damage. In an embodiment, at least a portion of the fiber optic cables 126 may not be disposed within the walls of the catheter 108. For example, at least a portion of the fiber optic cables 126 may be disposed within the central passageway 116 or on an external surface of the catheter 108 and/or the fingers 118. The fiber optic cables 126 that are not disposed within the walls of the catheter 108 or the fingers 118 may be attached to the catheter 108 or the fingers 118, for example, using an adhesive or a film covering the fiber optic cables 126. It is noted that a polymer or other biocompatible material may house the fiber optic cables 126, such as the fiber optic cables 126 that are disposed outside of the catheter 108, to protect the fiber optic cables 126. In an embodiment, the fiber optic cables 126 may be extruded with longitudinal joints. In such an example, the fiber optic cables 126 and the longitudinal joints form the outer periphery of the catheter 108 and/or form one or more circumferentially arranged rings.

In an embodiment, the laser debulking device 102 may include an optical light splitter (not shown). For example, the laser pulses 128 may be provided from a single source. The single source may include a single laser source or a single fiber optic cable. The optical light splitter may allow the laser pulses 128 from the single source to be split and provided to a plurality of laser emitters 120. For instance, the optical light splitter may allow the laser pulses 128 from the single laser source to be split and provided to a plurality of fiber optic cables 126 and the plurality of fiber optic cables 126 may provide the laser pulses 128 to a plurality of laser emitters 120.

As previously discussed, the fiber optic cables 126 may extend to the laser emitters 120. The laser emitters 120 are the portions of the laser debulking device 102 that emit the laser pulses 128. In an embodiment, the laser emitters 120 are the terminal ends of the fiber optic cables 126. In other words, the fiber optic cables 126 directly emit the laser pulses 128 from the terminal ends thereof. In an embodiment, the laser emitters 120 may include one or more lenses or other optical devices (e.g., at the terminal end of the fiber optic cables 126) that are configured to emit the laser pulses 128. In such an embodiment, the fiber optic cables 126 may provide the laser pulses 128 to the lenses or other optical devices and the lenses or other optical devices may emit the laser pulses 128. Generally, emitting the laser pulses 128 using the lenses or other optical devices produces a higher quality laser than emitting the laser pulses 128 from the fiber optic cables 126, for example, by focusing the laser pulses 128. However, due to the small size requirements of the laser debulking device 102 to allow the laser debulking device 102 to be positioned within the vascular structure, it may be difficult or impractical to install the lenses or other optical devices in the fingers 118.

The fingers 118 may include any suitable number of laser emitters 120. In an embodiment, as shown, each of the fingers 118 may include a single laser emitter 120. In an embodiment, at least one of the fingers 118 may include a plurality of laser emitters 120, such as 2-4, 3-5, 6-8, 7-10, 8-12, 10-15, 13-20, 15-25, 20-30, 25-40, 35-50, or more than 50. Generally, increasing the number of laser emitters 120 on each finger 118 may improve removal of the selected material by allowing more of the selected material to be irradiated at any given time. However, the number of laser emitters 120 may limited by the size of the fingers 118 (e.g., the maximum lateral dimension of the fingers 118 at the terminal end 122 thereof), the maximum lateral dimension of the laser emitters 120, the number of fiber optic cables 126 that may be included in the catheter 108, and other size restrictions.

The laser emitters 120 may emit the laser pulses 128 at an emittance angle θ relative to the central axis 114 of the catheter 108. In an embodiment, the emittance angle θ of the laser emitters 120 may be 0° (i.e., parallel), greater than 0° to about 5°, greater than 0° to about 1°, about 0.5° to about 1.5°, about 1° to about 2°, about 1.5° to about 3°, about 2° to about 4°, or about 3° to about 5°. It is noted that, as used herein, an emittance angle θ that is greater than zero extends at an angle away from the central longitudinal axis 114. The emittance angle θ of 0° or greater than 0° may be able to remove the selected material at or near the walls of the vascular structure. However, the emittance angle θ of 0° or greater than 0° may also cause damage to the vascular structure which, in some applications, may be undesirable. For instance, the laser pulses 128 emitted at an emittance angle θ of 0° or greater than 0° may diffuse slightly during use or local decreases in the maximum lateral dimension of the vascular structure (e.g., the vascular structure includes a protrusion), both of which may result in the laser pulses 128 irradiating and damaging the vascular structure. As such, in some embodiments and as shown in FIG. 1B, the laser emitters 120 may emit the laser pulses 128 at an emittance angle θ less than 0°, such as less than 0° to about −1°, about −0.5° to about −1.5°, about −1° to about −2°, about −1.5° to about −2.5°, about −2° to about −3°, about −2.5° to about −3.5°, than −3° to about −4°, about −3.5° to about −4.5°, about −4° to about −5°, about −4.5° to about −5°, or about −5° or less. It is noted that, as used herein, an emittance angle θ that is less than 0° extends at an angle oriented towards the central longitudinal axis 114 and FIG. 1B illustrates an emittance angle θ that is less than 0°. The emittance angle θ that is less than 0° may prevent or at least minimize the laser pulses 128 from irradiating the vascular structure during ordinary use thereby preventing damage to the vascular structure. The emittance angle θ that is less than 0° may also be less able to remove the selected material at or near the walls of the vascular structure than the emittance angle θ of 0° or greater than 0°. That said, the difference between removal of the selected material between the emittance angle θ that is less than 0° and the emittance angle θ that is 0° or greater than 0° may be negligible if the emittance angle θ is less than −5° or less than −3° since the laser emitters 120 may be positioned adjacent to the walls of the vascular structure when the fingers 118 are in the expanded state.

The laser emitters 120 may have maximum focal distance. As used herein, the maximum focal distance is the maximum distance from laser emitters 120 at which the laser pulses 128 may cut, burn, or otherwise effectively separate the selected material from the vascular structure during ordinary use. The maximum focal distance may be selected to be less than 5 cm, such as in ranges of about 100 μm to about 300 μm, about 200 μm to about 400 μm, about 300 μm to about 500 μm, about 400 μm to about 600 μm, about 500 μm to about 700 μm, about 600 μm to about 800 μm, about 700 μm to about 900 μm, about 800 μm to about 1 mm, about 900 μm to about 1.25 mm, about 1 mm to about 1.5 mm, about 1.25 mm to about 1.75 mm, about 1.5 mm to about 2 mm, about 1.75 mm to about 2.5, about 2 mm to about 3 mm, about 2.5 mm to about 3.5 mm, about 3 mm to about 5 mm, about 4 mm to about 6 mm, about 5 mm to about 7 mm, about 6 mm to about 8 mm, about 7 mm to about 9 mm, about 8 mm to about 1 cm, about 9 mm to about 1.25 cm, about 1 cm to about 1.5 cm, about 1.25 cm to about 1.75 cm, about 1.5 cm to about 2 cm, about 1.75 cm to about 2.5, about 2 cm to about 3 cm, about 2.5 cm to about 3.5 cm, or about 3 cm to about 5 cm. Generally, decreasing the maximum focal distance minimizes inadvertent damage to the walls of the vascular structure. However, decreasing the maximum focal distance may make removal of the selected material slower. As such, the maximum focal distance may be selected based on balancing these two factors and, as such, may vary from application to application (e.g., the size of the vascular structure or selected material to be removed) or from user to user of the laser debulking system 100 (e.g., based on user preference).

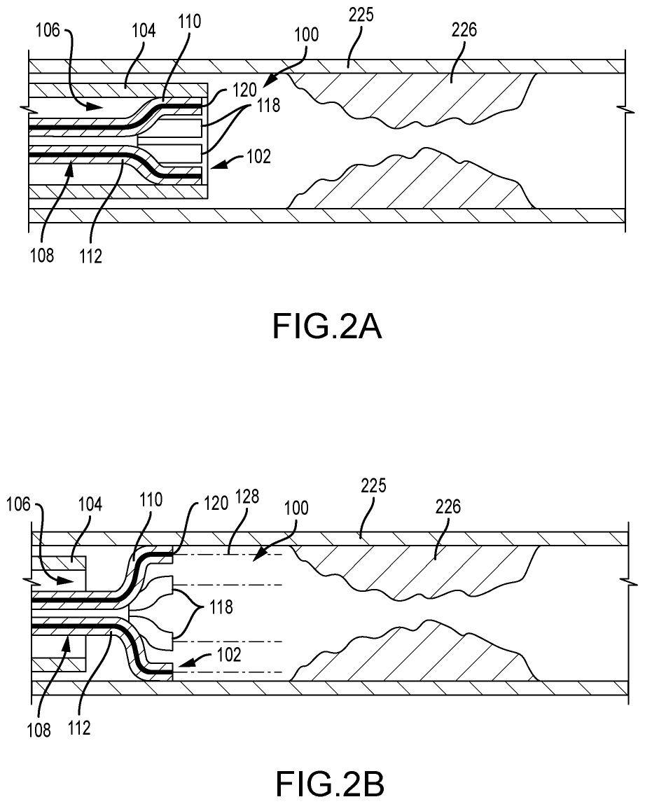

FIGS. 2A and 2B are cross-sectional views illustrating a method of using the laser debulking system 100, according to an embodiment. As shown in FIG. 2A, the laser debulking system 100 is positioned in a vascular structure 225 until the distal portions of the laser debulking system 100 are adjacent to a selected material 226. The vascular structure 225 may include any vessel in an individual, such as a vein, artery, or valve. The selected material 226 may include any material that is selected to be removed or otherwise separated from the vascular structure 225 using the laser debulking system 100. For example, the selected material 226 may include thrombus, scar tissue, an occlusion of an occluded venous stent, a venous stent, material received by or built up around an inferior vena cava filter, or any other material which may be desirable to be debulked and removed from the vascular structure.

The laser debulking system 100 may be positioned proximally relative to the selected material 226. That is, the laser debulking system 100 may be positioned on a side of the selected material 226 that is closer to an incision through which the laser debulking system 100 was inserted into the vascular structure 225. The fingers 118 may be in the deployment state when the laser debulking system 100 is moved in the in the vascular structure 225. For example, as shown, the fingers 118 may be positioned within the sheath 104.

After the distal end region 110 of the laser debulking system 100 is positioned proximate to the selected material 226, the fingers 118 of the laser debulking system 100 may be switched from the deployment state to the expanded state. For example, after positioning the laser debulking system 100, the sheath 104 may move proximally (e.g., away from the selected material 226) such that the fingers 118 are no longer covered by the sheath 104 thereby allowing the fingers 118 to expand. The fingers 118 may expand until the fingers 118 abut the vascular structure 225 which, in turn, prevents further expansion of the fingers 118. FIG. 2B illustrates the laser debulking system 100 in the expanded state.

With the fingers 118 in the expanded state, the laser debulking system 100 may emit a plurality of laser pulses 128 from the laser emitters 120 towards the selected material 426. The laser pulses 128 may irradiate the selected material 426 thereby causing the selected material 426 to be selectively detached from the vascular structure 225 or any other desired material (e.g., material attached to or otherwise associated with an occluded venous stent or an inferior vena cava filter). For example, the laser pulses 128 may be selected to vaporize, burn, or otherwise damage the selected material 226 thereby allowing the selected material 226 to be detached. It is noted that the laser pulses 128 do not need to irradiate all the selected material 426 to detach the selected material 226. For instance, the laser pulses 128 may be used to cut pieces of the selected material 226 from the vascular structure 225. In an example, while emitting the laser pulses 128, the laser debulking device 102 may be moved distally thereby allowing the laser debulking device 102 to remove more of the selected material 226.

In an example, while at least emitting the laser pulses 128, the laser debulking device 102 may be rotated generally about the central longitudinal axis 114 thereby allowing the laser debulking device 102 to remove all of the selected material 226 instead of just removing the portions of the selected material 226 that are initially in front of the laser emitters 120. The laser debulking device 100 may be configured to provide the ability for a user of the laser debulking device 100 (e.g., medical practitioner) the ability to rotate the fingers 118 at least one of clock-wise, counter clock-wise, a full rotation about the central longitudinal axis 114, or a partial rotation about the central longitudinal axis 114 as the laser debulking device 100 cuts, debulks, and/or moves relative to (e.g., advances through) the selected material 225. The rotation of the fingers 118 may be performed in a back and forth oscillatory direction or in a single directional rotational motion to cover completely the radial spacing between the laser emitters 120 to maximize radial coverage and efficiency of the debulking procedure.

After removing the selected material 226, the detached selected material 226 may be removed from the vascular structure 225. In an example, the laser debulking system 100 may be configured to provide aspiration to the vascular structure 225 and the aspiration may be used to remove the detached selected material 226 from the vascular structure 225. The aspiration may be provided, for instance, via the central passageway 116 of the laser debulking system 100. In an example, the detached selected material 226 may be removed from the vascular structure 225 using any other technique. For instance, the laser debulking system 100 may include a capture bag (not shown) positioned distally or downstream from the finger 118 and the capture bag is configured to receive and hold the detached selected material 226 therein. Any other techniques for removing the detached selected material 226, as known in the art or otherwise disclosed herein, may be used to remove the detached selected material 226 from the vascular structure 225.

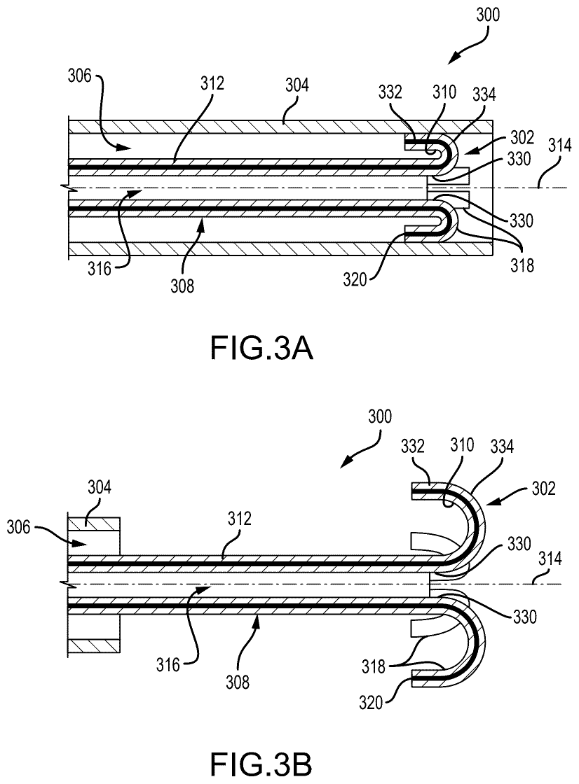

The laser debulking systems disclosed herein may exhibit structures other than the structure of the laser debulking system 100 illustrated in FIGS. 1A-2B. For example, FIGS. 3A and 3B are cross-sectional views of a laser debulking system 300 in the deployment and expanded states, respectively, according to an embodiment. Except as otherwise disclosed herein, the laser debulking system 300 is the same as or substantially similar to any of the laser debulking systems disclosed herein. For example, the laser debulking system 300 may include a laser debulking device 302 and a sheath 304. The laser debulking device 302 may include a catheter 308 including a distal end region 310 and a central region 312. The laser debulking device 302 also includes a plurality of finger 318 at the distal end region 310. At least one of the fingers 318 may include at least one laser emitter 320 that is configured to emit one or more laser pulses.

The fingers 118 of the laser debulking device 102 shown in FIGS. 1A-2B are configured to emit laser pulses in a distal direction. Unlike the fingers 118, the fingers 318 of the laser debulking device 302 shown in FIGS. 3A and 3B are configured to emit laser pulses in a proximal direction. As such, the laser emitters 320 may generally face in a proximal direction when the fingers 318 are in the expanded state.

In an embodiment, as shown, the fingers 318 include a first portion 330, a second portion 332, and a bend 334 therebetween. The first portion 330 may extend from the central region 312 of the catheter 308 in the distal direction and may be generally oriented parallel to the central longitudinal axis 314 of the catheter 308. The second portion 330 is spaced from the first portion 330 by the bend 334 and includes the laser emitter 320. When in the expanded configuration, the second portion 330 may extend parallel to the central longitudinal axis 314, extend from the bend 334 in the proximal direction, and be oriented such that the laser emitter 320 generally faces in the proximal direction. When the finger 318 is in the expanded state, the bend 334 between the first and second portions 330, 332 may be a generally 180° bend formed in the fingers 318 thereby allowing the first and second portions 330, 332 to exhibit the above-described orientations.

It is noted that the fingers 318 may include structures other than the first portion 330, the second portion 332, and the bend 334 that allows the laser debulking device 302 to emit the laser pulses in the proximal direction. In an embodiment, not shown, one or more of the first portion 330 or the second portion 332 are omitted from the fingers 318. In such an embodiment, the bend 334 extends directly from the central region 312 and/or the portion of the bend 334 furthest spaced from the central region 312 (when the bend 334 is bent straight and extends parallel to the central longitudinal axis 314) includes the laser emitter 320. In an embodiment, not shown, the fingers 318 may initially extend from the central region 312 at an oblique or perpendicular angular angle.

In an embodiment, the fingers 318 may be oriented such that the laser emitters 320 are oriented in the proximal direction when the fingers 318 are in the deployment state. For example, when in the deployment state (as shown in FIG. 3A) and in the expanded state (as shown in FIG. 3B), the fingers 318 may include the first portion 330 oriented parallel to the central longitudinal axis 314, the second portion 332 oriented parallel to the central longitudinal axis 314, and the bend 334.

It is noted that the fingers 318 may not be oriented such that the laser emitters 320 are oriented in proximal direction when the fingers 318 are in the deployment state. For example, FIG. 3C is a cross-sectional view of the laser debulking system 300 in the deployment state, according to an embodiment. As shown in FIG. 3C, the fingers 318 are oriented such that the laser emitters 320 are oriented in the distal direction when the fingers 318 are in the deployment state. Instead, the fingers 318 may extend in a direction that is generally parallel (e.g., ±30°) to the central longitudinal axis 314 when the fingers 318 are in the deployment state. When the fingers 318 include the bend 334, the bend 334 may be generally straightened when in the deployment state. However, the fingers 318 may exhibit sufficient flexibility that the fingers 318 in the deployment state shown in FIG. 3C are able to return to the expanded state, for instance, when the sheath 304 is not disposed over the fingers 318.

In an embodiment, the fingers 318 may exhibit both the deployment states shown in FIGS. 3A and 3C. For example, the fingers 318 may be initially provided in the deployment state shown in FIG. 3A. After removing the fingers 318 from the sheath 304 (e.g., moving the sheath 304 in a proximal direction), the fingers 318 may switch from the deployment state to the expanded state, such as the expanded state shown in FIG. 3B. However, switching the fingers 318 from the expanded state back to the deployment state (e.g., to facilitate removal of the laser debulking device 302 from the vascular structure) by advancing the sheath 304 distally may cause the sheath 304 to abut the bend 334. Further advancing the sheath 304 distally may force the fingers 318 to straighten out (e.g., the bend 334 to straighten out) and be disposed in the interior passageway 306 of the sheath 304, as shown in FIG. 3C.

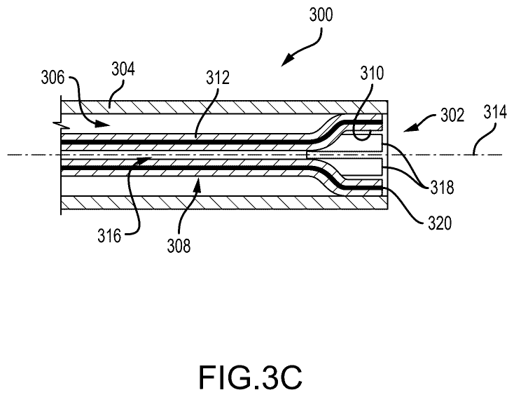

FIGS. 4A and 4B are cross-sectional views illustrating a method of using the laser debulking system 300, according to an embodiment. Except as otherwise disclosed herein, the method of using the laser debulking system 300 is the same as any of the other methods disclosed herein. As shown in FIG. 4A, the laser debulking system 300 is positioned in a vascular structure 425 until the distal portions of the laser debulking system 300 extend through the selected material 426 and the distal portions of the laser debulking system 300 are positioned distally from the selected material 426. That is, the laser debulking system 300 may be positioned on a side of the selected material 426 that is furthest from an incision through which the laser debulking system 300 was inserted into the vascular structure 425. The fingers 318 may be in the deployment state when the laser debulking system 300 is moved in the vascular structure 425. For example, as shown, the fingers 318 may be positioned within the sheath 304.

After the distal end region 310 of the laser debulking system 300 is positioned distal to the selected material 426, the fingers 318 of the laser debulking system 300 may be switched from the deployment state to the expanded state. For example, after positioning the laser debulking system 300, the sheath 304 may move proximally such that the fingers 318 are no longer covered by the sheath 304 thereby allowing the fingers 318 to expand. The fingers 318 may expand until the fingers 318 abut the vascular structure 425 which, in turn, prevents further expansion of the fingers 318. FIG. 4B illustrates the laser debulking system 300 in the expanded state.

With the fingers 318 in the expanded state, the laser debulking system 300 may emit a plurality of laser pulses 428 from the laser emitters 320 towards the selected material 426. The laser pulses 428 may irradiate the selected material 426 thereby causing the selected material 426 to be selectively detached from the vascular structure 425 or any other desired material (e.g., material attached to or otherwise associated with an occluded venous stent or an inferior vena cava filter). The laser debulking system 300 may move proximally and/or rotate as the laser emitters 320 emit the laser pulses 428. After removing the selected material 426, the detached selected material 426 may be removed from the vascular structure 425 using any of the method disclosed herein.

Whether the laser debulking system 100 shown in FIGS. 1A-2B or the laser debulking system 300 shown in FIGS. 3A-4B are used to remove the selected material from the vascular structure may be selected for a variety of reasons. In an example, whether the laser debulking system 100 or the laser debulking system 300 is used may be selected based on preference of the medical practitioner performing the procedure. In an example, whether the laser debulking system 100 or the laser debulking system 300 is used may be selected based on the selected material to be removed. As shown in FIGS. 4A and 4B, the laser debulking system 300 typically extends through the selected material before being used. As such, the laser debulking system 300 may only be used when the laser debulking system 300 may easily extend through the selected material without forming one or more emboli or otherwise damaging the vascular structure. In an example, whether the laser debulking system 100 or the laser debulking system 300 is used may be selected depending on whether the vascular structure includes a medical device (e.g., a stent, inferior vena cava filter, etc.) therein and whether moving the laser debulking system 100 or the laser debulking system 300, in either the collapsed or expanded state, may become entangled with, inadvertently dislodge, or otherwise damage the medical device. In such an example, the structure of the medical device may dictate whether the laser debulking system 100 or the laser debulking system 300 is used. In an example, whether the laser debulking system 100 or the laser debulking system 300 is used may be selected based on the direction of the flow of blood or other fluids in the vascular structure. For instance, the laser debulking systems 100 and 300 may be configured to provide aspiration to the vascular structure to remove the detached selected material using the interior passageway, the central passageway, or other structure. When the flow of blood or other fluids in the vascular structure is in a proximal direction, the flow of blood or other fluids may move the detached selected material towards the aspiration sources of laser debulking system 100 (e.g., the inlet of the interior passageway 106 or the inlet of the central passageway 116) thereby increasing the likelihood that the aspiration provided by the laser debulking system 100 receives substantially all of the detached selected material and decreases the likelihood that the detached selected material forms one or more emboli. When the flow of blood or other fluids in the vascular structure is in a distal direction, the flow of blood or other fluids may move the detached selected material towards at least one of aspiration sources of the laser debulking system 300 (e.g., the inlet of the central passageway 316) thereby increasing the likelihood that the aspiration provided by the laser debulking system 300 receives the detached selected material and decreases the likelihood that the detached selected material forms one or more emboli.

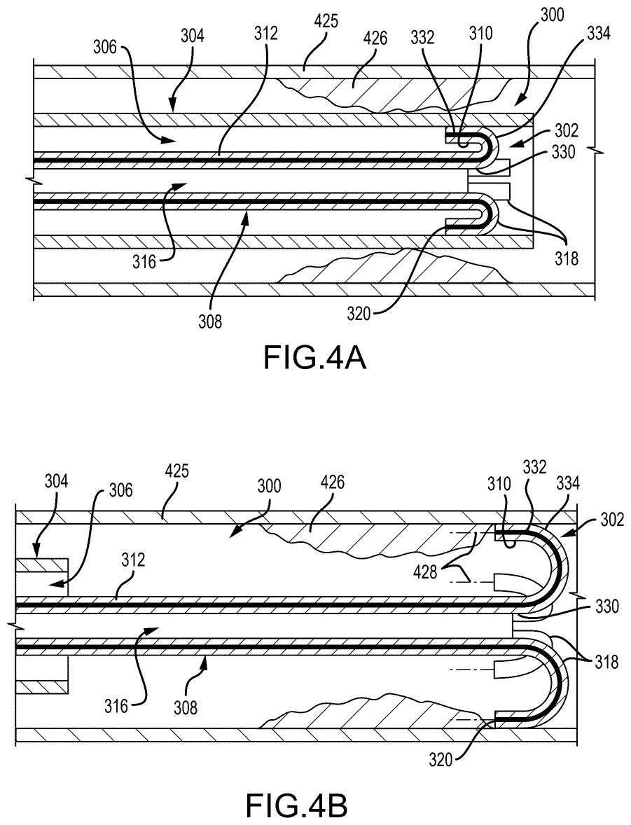

The laser debulking devices disclosed herein may include features other than those discussed above. For example, FIG. 5 is a front view of a laser debulking device 502 including one or more aspiration channels 536, according to an embodiment. Except as otherwise disclosed herein, the laser debulking device 502 may be the same as or substantially similar to any of the laser debulking devices disclosed herein. For example, the laser debulking device 502 may include a catheter 508 defining a central passageway 516, a plurality of fingers 518 (shown in the expanded state), and one or more laser emitters 520.

The laser debulking device 502 defines one or more aspiration channels 536 that are distinct from the central passageway 516. The aspiration channels 536 are configured to provide aspiration to the vascular structure to remove detached selected material from the vascular structure. For example, the aspiration channels 536 may extend at least substantially all of a length of the catheter 508 and be connected to a vacuum source. The aspiration channels 536 that are distinct from the central passageway 516 allows the central passageway 516 to have one or more devices (e.g., guidewire) disposed therein while still being able to provide the aspiration to the vascular structure.

The laser debulking device 502 may include any suitable number of aspiration channels 536. For example, as shown, the laser debulking device 502 includes an equal number of aspiration channels 536 as fingers 518. That said, the laser debulking device 502 may include fewer or more aspiration channels 536 than fingers 518. Generally, decreasing the number of aspiration channels 536 allows the size of the aspiration channels 536 to be increased so long as the thickness of the aspiration channels 536 is less than a thickness of the catheter 508, wherein the thickness of the aspiration channels 536 and the catheter 508 are measured radially relative to the central longitudinal axis (not shown).

It is noted that the aspiration channels 536 are illustrated as being arranged circumferentially around the longitudinal central axis. However, it is noted that the aspiration channels 536 may exhibit other arrangements or shapes. In an example, the aspiration channels 536 may only be formed on one side or more concentrated on one side of the central longitudinal axis. In an example, the aspiration channels 536 may include a single aspiration channel, such as a generally crescent shaped aspiration channel 536.

In an embodiment, the aspiration channels 536 are in a portion of the catheter 508 between the fingers 518. Such positioning of the aspiration channels 536 allows the aspiration channels 536 and the fiber optic cables to extend generally parallel to the central longitudinal axis without interfering with each other. The aspiration channels 536 and the fiber optic cables extending generally parallel to the central longitudinal axis may allow the catheter 508 to be at least partially formed using a coextrusion technique.

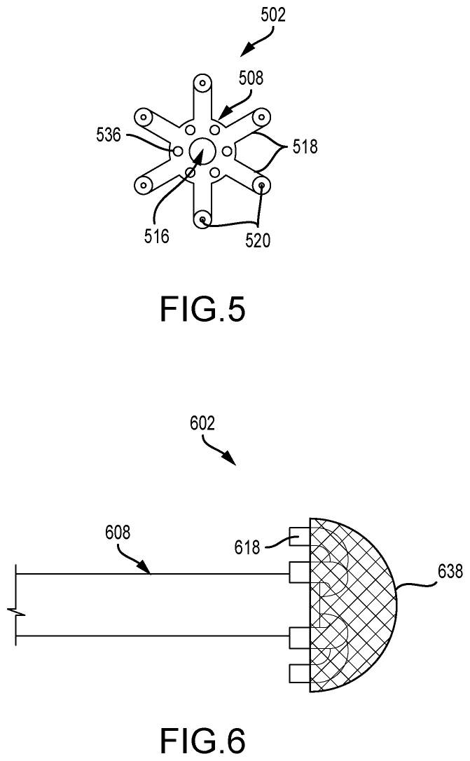

FIG. 6 is a side view of a laser debulking device 602 including a capture bag 638, according to an embodiment. Except as otherwise disclosed herein, the laser debulking device 602 is the same as or substantially similar to any of the laser debulking devices disclosed herein. For example, the laser debulking device 602 may include a catheter 608 defining a central passageway, a plurality of fingers 618 (shown in the expanded state), and one or more laser emitters.

The laser debulking device 602 includes the capture bag 638. The capture bag 638 may be a mesh bag or other porous material defining a plurality of gaps therein that allows blood or other bodily fluids to pass therethrough while substantially preventing the detached selected material from passing therethrough. As such, the capture bag 638 is configured to receive and hold the detached material thereby minimizing the risk of forming one or more emboli. It is noted that the capture bag 638 may switch between a deployment state (e.g., the capture bag 638 is disposed in a sheath) and an expanded state, as shown. The capture bag 638 is generally positioned downstream of the selected material. In an example, as shown, the capture bag 638 is attached to and extends from the fingers 618 when the fingers 618 are positioned downstream of the selected material. In an example, the capture bag 638 may include a nickel-titanium alloy that is configured to expand when deployed from the central passageway. In an embodiment, the laser debulking device 602 may include an embolic cap instead of or in addition to the capture bag 638.

FIG. 7 is a schematic side view of a laser debulking system 700, according to an embodiment. Except as otherwise disclosed herein, the laser debulking system 700 may be the same as or substantially similar to any of the laser debulking systems disclosed herein and may include any of the laser debulking devices disclosed herein. The laser debulking system 700 includes a catheter 708. The catheter 708 may include a proximal end region 741 and a distal end region 710. The proximal end region 741 may be attached to a handle 740 and the distal end region 710 may include a plurality of fingers 718. The handle 740 may be configured to move the catheter 708 when the catheter 708 is inserted into an individual. For example, moving the handle 740 in a distal direction may advance the catheter 708 into an individual and moving the handle 740 in a proximal direction may retract the catheter 708 from the individual. It is noted that the handle 740 may exhibit any structure known in the art and may move (e.g., proximally, distally, or rotate) the catheter 708 using any conventional technique.

The laser debulking system 700 may also include a sheath 704 configured to switch the fingers 718 between a deployment state and an expanded state. The sheath 704 is configured to slide along the catheter 708. Sliding the sheath 704 in a distal direction may cause the sheath 704 to receive the fingers 718. Receiving the fingers 718 into the sheath 704 may cause the fingers 718 to be in the deployment state by forcing the maximum lateral dimension of the fingers 718 to decrease until the maximum lateral dimension of the fingers 718 is small enough to fit within the sheath 704. For example, the fingers 718 are radially collapsible to a smaller diameter to be contained at least partially within the sheath 704 for insertion and retrieval. Sliding the sheath 704 in a proximal direction may cause the sheath 704 to expose the fingers 718, thereby allowing the fingers 718 to switch from the deployment state to the expanded state. Exposing the fingers 718 allows the maximum lateral dimension of the fingers 718 to increase. In an embodiment, the laser debulking system 700 may include an actuator 742 that, when actuated, causes the sheath 704 to move in the distal direction or the proximal direction. The actuator 742 may be distinct (e.g., spaced) from the handle 740 (as shown) or form part of the handle 740.

The laser debulking system 700 may include a laser source 744. The laser source 744 is configured to generate the laser pulses. The laser debulking system 700 is configured to transmit the laser pulses generated by the laser source 744 to the laser emitters. For example, the laser debulking system 700 may include one or more fiber optic cables 746. The fiber optic cables 746 may be positioned and configured to receive the laser pulses generated by the laser source 744 and transmit (either directly or indirectly) the laser pulses to the laser emitters. For instance, the fiber optic cables 746 may extend from the laser source 744, to the handle 740, into the catheter 708, and to the laser emitters.

In an embodiment, the laser debulking system 700 may include a vacuum source 748 configured to provide aspiration to the vascular structure during use of the laser debulking system 700. For example, the laser debulking system 700 may include one or more conduits 750 that fluidly couple the vacuum source 748 with the catheter 708. For example, the conduits 750 may fluidly connect the vacuum source 748 with the handle 740. The vacuum source 748 may include a manual vacuum pump, and electric vacuum pump, a diaphragm pump, a centrifugal pump, a displacement pump, a magnetically driven pump, a peristaltic pump, a wall-mounted vacuum source, or any pump configured to produce a vacuum. In an embodiment, the vacuum source 748 may include a syringe, wherein extraction of a plunger of the syringe provides the aspiration.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. A laser debulking device for removing a selected material from vasculature of a subject, the laser debulking device, comprising:
a catheter including:
a proximal end region; and
a distal end region opposite the proximal end region;
a plurality of fingers integrally formed with or attached to the distal end region, the plurality of fingers configured to switch between a deployment state and an expanded state, wherein a maximum lateral dimension of the plurality of fingers is greater when in the expanded state than the deployment state; and
a plurality of laser emitters, at least some of the plurality of fingers including at least one of the plurality of laser emitters;
wherein, when in the expanded state, the plurality of fingers including a first bend at or near the distal end region that at least initially bends away from a central longitudinal axis of the catheter, the first bend causing the plurality of laser emitters to emit laser pulses generally parallel to the central longitudinal axis or the plurality of fingers including a second bend that causes the plurality of laser emitters to emit laser pulses generally parallel to the central longitudinal axis.

2. The laser debulking device of claim 1, wherein at least a portion of the plurality of fingers extend in a generally distal direction when in the deployment state, the distal direction extending along the central longitudinal axis of the catheter from the proximal end region to the distal end region.

3. The laser debulking device of claim 1, wherein the plurality of laser emitters are configured to emit the laser pulses generally in a distal direction, the distal direction extending along the central longitudinal axis of the catheter from the proximal end region to the distal end region.

4. The laser debulking device of claim 1, wherein at least a portion of the plurality of fingers extend generally in a proximal direction when in the deployment state, the proximal direction extending along the central longitudinal axis of the catheter from the distal end region to the proximal end region.

5. The laser debulking device of claim 1, wherein the plurality of laser emitters are configured to emit the laser pulses generally in a proximal direction, the proximal direction extending along the central longitudinal axis of the catheter from the distal end region to the proximal end region.

6. The laser debulking device of claim 5, wherein the first bend is a generally 180° bend.

7. The laser debulking device of claim 1, wherein the plurality of laser emitters are configured to emit laser pulses therefrom in an emittance direction when the plurality of fingers are in the expanded state, the emittance direction extending at an angle of less than 0° to about −5° relative to the central longitudinal axis of the catheter.

8. The laser debulking device of claim 1, wherein the distal end region of the catheter defines one or more aspiration channels configured to provide aspiration at or near the distal end region of the catheter.

9. The laser debulking device of claim 8, wherein the one or more aspiration channels includes a plurality of aspiration channels, each of the plurality of aspiration channels positioned between adjacent fingers of the plurality of fingers.

10. The laser debulking device of claim 1, wherein the plurality of laser emitters are connected to or are formed by a plurality of fiber optic cables.

11. The laser debulking device of claim 1, further comprising a capture bag configured to receive and hold removed material.

12. The laser debulking device of claim 11, wherein the capture bag is attached to at least some of the plurality of fingers.

13. The laser debulking device of claim 11, wherein the capture bag is positioned distally relative to the plurality of fingers.

14. A laser thrombus-debulking system, comprising:
a laser debulking device, including:
a catheter including:
a proximal end region; and
a distal end region opposite the proximal end region;
a plurality of fingers integrally formed with or attached to the distal end region, the plurality of fingers configured to switch between a deployment state and an expanded state, wherein a maximum lateral dimension of the plurality of fingers is greater when in the expanded state than the deployment state; and
a plurality of laser emitters, at least some of the plurality of fingers including at least one of the plurality of laser emitters;
wherein, when in the expanded state, the plurality of fingers including a first bend at or near the distal end region that at least initially bends away from a central longitudinal axis of the catheter, the first bend causing the plurality of laser emitters to emit laser pulses generally parallel to the central longitudinal axis or the plurality of fingers including a second bend that causes the plurality of laser emitters to emit laser pulses generally parallel to the central longitudinal axis; and
a sheath defining interior passageway, the catheter of the laser debulking device at least partially disposed in the interior passageway, the sheath configured to slide along the catheter, and wherein the plurality of fingers are in the deployment state when the plurality of fingers are disposed in the interior passageway and are in the expanded state when the plurality of fingers are at least partially disposed outside of the interior passageway.

15. The laser debulking system of claim 14, further comprising:
a laser source; and
a plurality of optical fiber cables configured to receive laser pulses generated by the laser source, the plurality of optical fiber cables coupled to the plurality of laser emitters.

16. A method of using a laser debulking device, the method comprising:
positioning a distal end region of a catheter adjacent to or through a selected material in a vasculature of a subject to be debulked, the selected material to be debulked located within a vascular structure;
switching a plurality of fingers from a deployment state to an expanded state, the plurality of fingers attached to or forming the distal end region; and
with the plurality of fingers in the expanded state, emitting a plurality of laser pulses from a plurality of laser emitters towards the selected material to be debulked, at least some of the plurality of fingers including at least one of the plurality of laser emitters;
wherein, when in the expanded state, the plurality of fingers including a first bend at or near the distal end region that at least initially bends away from a central longitudinal axis of the catheter, the first bend causing the plurality of laser emitters to emit laser pulses generally parallel to the central longitudinal axis or the plurality of fingers including a second bend that causes the plurality of laser emitters to emit laser pulses generally parallel to the central longitudinal axis.

17. The method of claim 16, wherein emitting a plurality of laser pulses includes rotating the plurality of fingers as the plurality of laser pulses are emitted.

18. The method of claim 16, wherein the material to be debulked includes a thrombus or scar tissue.

19. The method of claim 16, wherein the material to be debulked includes an occlusion in an occluded venous stent.

20. The method of claim 16, wherein the material to be debulked includes tissue within or adjacent to an inferior vena cava filter.

21. The laser debulking device of claim 1, wherein, when in the expanded state, the plurality of fingers includes the first bend and the second bend.
